(19) United States
(12) Patent Application Publication
(10) Pub. No.: US 2024/0387016 A1
(43) Pub. Date: Nov. 21, 2024

(54) ASSESSMENT EVALUATION DEVICE, ASSESSMENT SUPPORT METHOD, AND STORAGE MEDIUM

(71) Applicant: NEC Corporation, Minato-ku, Tokyo (JP)

(72) Inventors: Kenji ARAKI, Tokyo (JP); Yutaka UNO, Tokyo (JP); Junichi YAHARA, Tokyo (JP); Yuki KUSANO, Tokyo (JP); Kento SOMA, Tokyo (JP)

(73) Assignee: NEC Corporation, Minato-ku, Tokyo (JP)

(21) Appl. No.: 18/692,872

(22) PCT Filed: Sep. 28, 2021
(86) PCT No.: PCT/JP2021/035573

§ 371 Date: Mar. 18, 2024

(51) Int. Cl. G16H 15/00; G06F 40/30

(52) U.S. Cl. CPC G16H 15/00; G06F 40/30

(57) ABSTRACT

The assessment evaluation device 1X mainly includes a described word vector generation means 17X, an ideal word vector generation means 15X, and an evaluation means 18X. The described word vector generation means 17X generates, based on a nursing assessment of a target patient whose nursing records are to be prepared, a described word vector which is a word vector obtained by vectorizing whether or not each of predetermined candidate words is described in the nursing assessment. The ideal word vector generation means 15X generates an ideal word vector that is an ideal of the word vector based on a patient condition of the target patient. The evaluation means 18X determines an evaluation of the nursing assessment based on the described word vector and the ideal word vector. It can be used to support user's decision making.

### TECHNICAL FIELD

The present disclosure relates to the technical field of the assessment evaluation device, the assessment evaluation method, and the storage medium configured to evaluate the assessment (nursing assessment) which is a part of nursing records.

### BACKGROUND

As a system for the support of the medical treatment and treatment policy, there is known a system which defines the degree of similarity by using a symptom vector obtained by quantifying the symptom of a target patient and detects a past patient whose symptom vector is similar to the target patient (e.g., see Patent Literature 1).

### CITATION LIST

### Patent Literature

Patent Literature 1: JP2003-122845A

### SUMMARY

### Problem to be Solved

The assessment described as one item of the nursing records requires analysis on the nursing policy of the target patient and description of the thought process thereof, and it is difficult for nurses with little experience to accurately describe the assessment. Therefore, a system for evaluating whether or not the assessment is described correctly is required to support the preparation of appropriate nursing records.

In view of the above-described issue, it is therefore an example object of the present disclosure to provide an assessment evaluation device, assessment evaluation method, and storage medium capable of evaluating the assessment.

### Means for Solving the Problem

One aspect of the assessment evaluation device is an assessment evaluation device including:
a described word vector generation means configured to generate, based on a nursing assessment of a target patient whose nursing records are to be prepared, a described word vector which is a word vector obtained by vectorizing whether or not each of predetermined candidate words is described in the nursing assessment;
an ideal word vector generation means configured to generate an ideal word vector that is an ideal of the word vector based on a patient condition of the target patient; and
an evaluation means configured to determine an evaluation of the nursing assessment based on the described word vector and the ideal word vector.

One aspect of the assessment evaluation method is an assessment evaluation method executed by a computer, the assessment evaluation method including:
generating, based on a nursing assessment of a target patient whose nursing records are to be prepared, a described word vector which is a word vector obtained by vectorizing whether or not each of predetermined candidate words is described in the nursing assessment;
generating an ideal word vector that is an ideal of the word vector based on a patient condition of the target patient; and
determining an evaluation of the nursing assessment based on the described word vector and the ideal word vector.

One aspect of the storage medium is a storage medium storing a program executed by a computer, the program causing the computer to:
generate, based on a nursing assessment of a target patient whose nursing records are to be prepared, a described word vector which is a word vector obtained by vectorizing whether or not each of predetermined candidate words is described in the nursing assessment;
generate an ideal word vector that is an ideal of the word vector based on a patient condition of the target patient; and
determine an evaluation of the nursing assessment based on the described word vector and the ideal word vector.

### Effect

An example advantage according to the present invention is to suitably determine an evaluation of a nursing assessment of a target patient.

### BRIEF DESCRIPTION OF THE DRAWINGS

FIG. 1 It shows a schematic configuration of an assessment support system according to the first example embodiment.

FIG. 2A is a diagram showing a process outline of an assessment evaluation device.

FIG. 2B is a diagram showing an outline of a process according to a regression model.

FIG. 3A illustrates the hardware configuration of the assessment evaluation device.

FIG. 3B illustrates the hardware configuration of a learning device.

FIG. 4 It is an example of a functional block of the assessment evaluation device.

FIG. 5 It is an example of a functional block of the learning device.

FIG. 6 It is a diagram showing a processing overview of the assessment evaluation device in a specific example.

FIG. 7 It illustrates the first display example on the evaluation of the assessment.

FIG. 8 It illustrates the second display example on the evaluation of the assessment.

FIG. 9 It illustrates the third display example on the evaluation of the assessment.

FIG. 10 It illustrates an example of a flowchart to be executed by the assessment evaluation device in the first example embodiment.

FIG. 11 It illustrates a schematic configuration of an assessment support system according to a second example embodiment.

FIG. 12 It illustrates an example of functional blocks of the assessment evaluation device.

FIG. 13 It illustrates an example of a flowchart to be executed by the assessment evaluation device in the second example embodiment.

FIG. 14 It illustrates a schematic configuration of an assessment support system in the third example embodiment.

FIG. 15 It is a block diagram of an assessment evaluation device according to a fourth example embodiment.

FIG. 16 It is an example of a flowchart to be executed by the assessment evaluation device in the fourth example embodiment.

### EXAMPLE EMBODIMENTS

Hereinafter, example embodiments of an assessment evaluation device, an assessment support method, and a storage medium will be described with reference to the drawings.

### First Example Embodiment

### (1) System Configuration

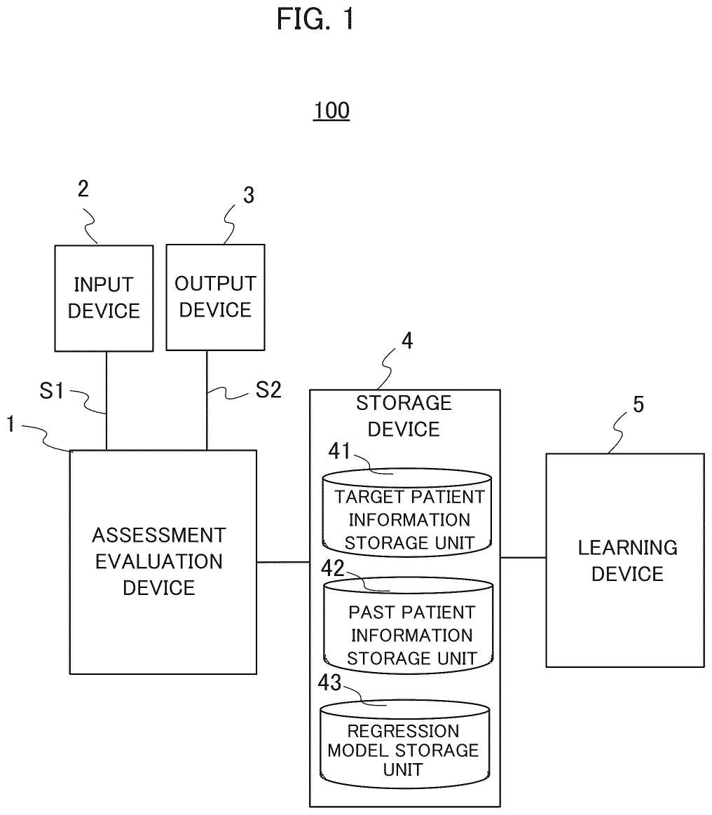

FIG. 1 shows a schematic configuration of an assessment support system 100 according to a first example embodiment. The assessment support system 100 is a system for supporting the preparation of an assessment (in detail, a nursing assessment) by a medical worker (typically, a nurse, a nursing student, or the like; hereinafter, referred to as “user”). Here, the term “assessment” is a description item included in the “SOAP” which is one of analytical methods regarding nursing records recorded for nursing patients. Among “SOAP”, “S” indicates subjective information regarding a patient, “O” indicates objective information regarding the patient, “A” indicates the “assessment”, and “P” indicates a nursing plan. In the “assessment”, there are described a judgment, an evaluation, an opinion and an impressions and the like derived by analyzing subjective information (i.e., “S”) and objective information (i.e., “O”). The sentence (text) information described as the “assessment” in nursing records is referred to as “assessment information” in particular.

The assessment support system 100 mainly includes an assessment evaluation device 1, an input device 2, an output device 3, a storage device 4, and a learning device 5.

The assessment evaluation device 1 evaluates the assessment designated as an evaluation target. The assessment evaluation device 1 performs data communication with the input device 2, the output device 3, and the storage device 4 through a communication network or through wireless or wired direct communication. Then, based on the input signal “S1” supplied from the input device 2 and the information stored in the storage device 4, the assessment evaluation device 1 identifies and evaluates the target assessment of the evaluation. The assessment evaluation device 1 generates an output signal “S2” related to the evaluation result of the assessment, and supplies the generated output signal S2 to the output device 3.

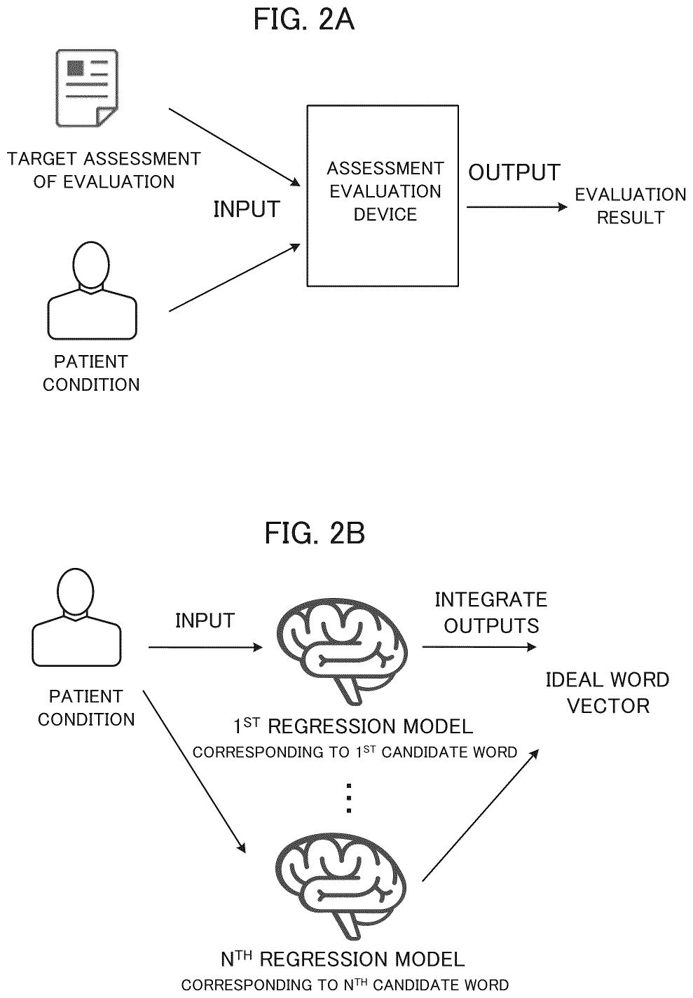

FIG. 2A is a diagram showing a process outline of the assessment evaluation device 1. In summary, the assessment evaluation device 1 determines an evaluation of the assessment based on the target assessment of the evaluation expressed as a text and the condition of a target patient of the assessment, and outputs the evaluation result indicating the determined evaluation. In this case, the assessment is converted into a word vector obtained by quantifying the presence or absence of each of predetermined words (also referred to as “candidate words”) serving as keywords in the generation of the assessment. A candidate word is a candidate for a word that is necessary to be included in the target assessment of evaluation, and a plurality of words are defined as candidate words. Examples of the above-described candidate words and the patient conditions will be described later.

A description will be given of the configuration other than the assessment evaluation device 1 with reference to FIG. 1.

The input device 2 is an interface that accepts manual input (external input) of information by the user. Examples of the input device 2 include a variety of user input interfaces such as, for example, a touch panel, a button, a keyboard, a mouse, and a voice input device. The input device 2 supplies the generated input signal S1 to the assessment evaluation device 1. The output device 3 displays or output, by audio, a predetermined information, based on the output signal S2 supplied from the assessment evaluation device 1. Examples of the output device 3 include a display, a projector, and a speaker.

The storage device 4 is a memory configured to store various information necessary for processing performed by the assessment evaluation device 1 and the learning device 5. The storage device 4 may be an external storage device such as a hard disk connected or embedded in the assessment evaluation device 1 or the learning device 5, or may be a storage medium such as a flash memory. The storage device 4 may be a server device that performs data communication with the assessment evaluation device 1 and the learning device 5. The storage device 4 may be configured by a plurality of devices. Further, at least a part of the information stored in the storage device 4 may be stored by the assessment evaluation device 1 or the learning device 5.

The storage device 4 functionally includes a target patient information storage unit 41, a past patient information storage unit 42, and a regression model storage unit 43.

The target patient information storage unit 41 stores target patient information which is information regarding a patient (also referred to as “target patient”) whose assessment is to be evaluated. The target patient information includes information regarding the patient condition of the target patient. The information regarding the patient condition of the target patient includes at least one of attribute information representing basic attributes of the target patient and examination information indicating the previous examination results of the target patient and the nursing record information which is information on nursing records in the past certain period of the target patient. The target patient information storage unit 41 may further store nursing records on the current medical condition of the target patient.

Here, examples of the attribute information include age, gender, disease name, disease stage, nursing diagnosis, and past medical history. Examples of the examination information include height, weight, vital, and blood examination results. Examples of the nursing record information is a word vector generated from nursing records (especially assessment information) of the target patient during a certain period of the past. In this case, the word vector may be a word vector extracted by preprocessing the assessment information of the target patient generated in a past certain period, or may be a word vector obtained by converting the above assessment information using a technique for vectorizing a sentence. The former word vector is, for example, a vector representing the presence or absence of each of the candidate words in the past assessment data of the target patient, and the latter word vector is a word vector generated based on any sentence vectorization technique such as BoW (Bag of Words) and Word2vec.

The past patient information storage unit 42 stores the past patient information which is information regarding patients (also referred to as “past patients”) whose assessments are generated in the past and recorded in the nursing records. The assessments of past patients were appropriately generated, for example, by veteran medical workers. The past patient information storage unit 42 stores past patient information regarding multiple past patients. The past patient information at least includes past assessment information indicating the assessment generated in the past and past patient condition information indicating the patient conditions of the past patients at the time of generation of the assessments. The past patient information is used for learning of regression models described later.

The information stored in the target patient information storage unit 41 and the past patient information storage unit 42 may be a database stored as patient information for a plurality of patients or various data generated and organized to be available for other systems (e.g., electronic medical record system). In this case, the patient information is information recorded for each patient (i.e., known information), and may include, for example, medical records generated by a doctor for each patient, examination data, and nursing records. Such patient information is associated with, for example, patient identification information (also referred to as “patient ID”) for identifying each patient. Then, the information stored by the target patient information storage unit 41 and the past patient information storage unit 42, instead of being divided into two and dispersedly managed, may be managed as one database, or managed as three or more databases.

The regression model storage unit 43 stores information on one or more regression models configured to output, in a regressive manner, information regarding words (also referred to as “ideal words”) that are necessary to be included (i.e., ideal to be included) in the assessment of the target patient of evaluation. For example, a regression model is provided for each candidate word and is trained to output the probability that the corresponding candidate word is an ideal word (i.e., the probability that the candidate word appears in the ideal assessment) when information indicating the patient condition is inputted thereto. In other words, the regression model is a model that has learned the relation between a patient condition and whether or not the description of a candidate word in the assessment is necessary. Such a regression model may be a statistical model, such as a logistic regression model, or a machine learning model, such as a neural network. Then, the regression model storage unit 43 stores the information regarding the parameters necessary for configuring the learned regression model. For example, when the above-described regression model is a model based on a neural network such as a convolutional neural network, the regression model storage unit 43 stores information regarding various parameters such as the layer structure, the neuron structure of each layer, the number of filters and the filter size in each layer, and the weight for each element of each filter.

The learning device 5 trains the regression model based on the past patient information stored in the past patient information storage unit 42, and stores the learned parameters of the regression model in the regression model storage unit 43. The learning device 5 trains the regression model in advance before the execution of evaluating the assessment by the assessment evaluation device 1.

FIG. 2B is a diagram illustrating an overview of the process performed by the regression model trained by the learning device 5. In this example, there are N candidate words (N is an integer of 2 or more), and N regression models (first regression model to Nth regression model) for candidate words (first candidate word to Nth candidate word) have been trained, respectively. Then, when information indicating the patient condition of the target patient is inputted to each regression model, each regression model outputs the likelihood (probability) that the candidate word is an ideal word. Then, the assessment evaluation device 1 integrates the outputs from the regression models to generate an N-dimensional word vector (also referred to as “ideal word vector”) indicating whether or not the candidate word for each dimension is an ideal word. Then, as will be described later, based on the comparison result between the ideal word vector and a word vector (also referred to as “described word vector”) indicating whether or not each ideal word is actually described in the target assessment of the evaluation, the assessment evaluation device 1 determines the evaluation of the assessment. Thus, the assessment evaluation device 1 determines the evaluation of the target assessment of the evaluation based on the presence or absence of the description of each ideal word that is identified based on the patient condition.

The configuration of the assessment support system 100 shown in FIG. 1 is an example, and various changes may be made to the configuration. For example, the assessment evaluation device 1 and the learning device 5 may be realized by a single device, and evaluation of the assessment and learning of the regression model may be performed by the single device. In other examples, the input device 2 and the output device 3 may be integrally configured. In this case, the input device 2 and the output device 3 may be configured as a tablet-type terminal that is integral to or separate from the assessment evaluation device 1. The assessment evaluation device 1 may be configured by a plurality of devices. In this case, the plurality of devices constituting the assessment evaluation device 1 exchange information necessary to execute the process assigned in advance with one another. In this case, the assessment evaluation device 1 functions as a system.

### (2) Hardware Configuration

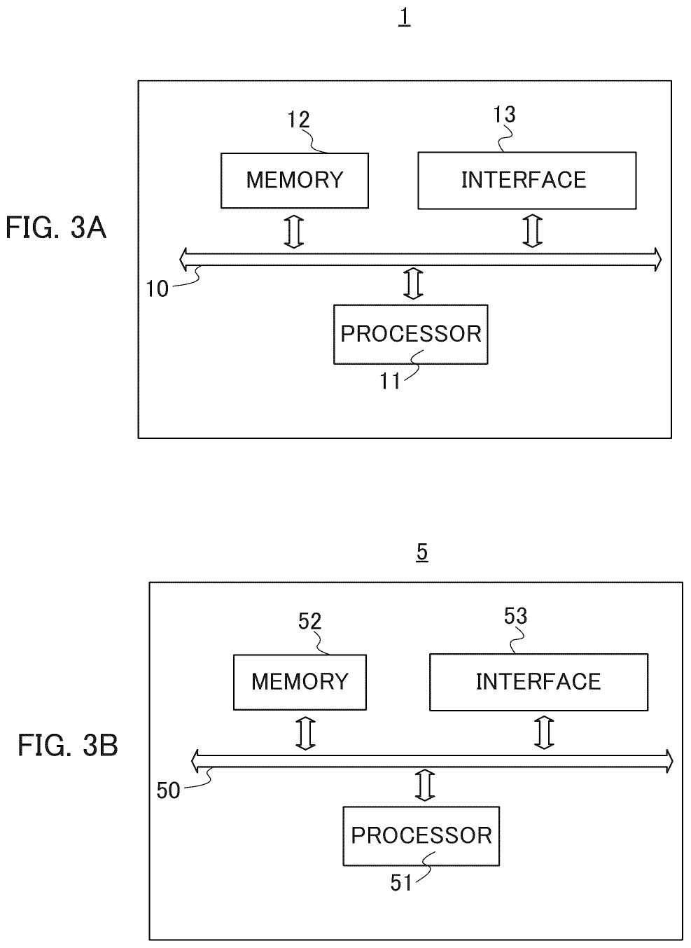

FIG. 3A shows a hardware configuration of the assessment evaluation device 1. The assessment evaluation device 1 includes, as hardware, a processor 11, a memory 12 and an interface 13. The processor 11, the memory 12 and the interface 13 are connected to one another via a data bus 10.

The processor 11 functions as a controller (arithmetic unit) for controlling the entire assessment evaluation device 1 by executing a program stored in the memory 12. Examples of the processor 11 include a CPU (Central Processing Unit), a GPU (Graphics Processing Unit), a TPU (Tensor Processing Unit) and the like. The processor 11 may be configured by a plurality of processors. The processor 11 is an example of a computer.

The memory 12 is configured by a variety of volatile and non-volatile memories, such as a RAM (Random Access Memory), a ROM (Read Only Memory), and a flash memory. Further, the memory 12 stores a program for executing a process performed by the assessment evaluation device 1. Apart of the information stored in the memory 12 may be stored in one or more external storage devices capable of communicating with the assessment evaluation device 1, or may be stored in a storage medium detachable from the assessment evaluation device 1.

The interface 13 is one or more interfaces for electrically connecting the assessment evaluation device 1 to other devices. Examples of these interfaces include a wireless interface, such as a network adapter, for transmitting and receiving data to and from other devices wirelessly, and a hardware interface, such as a cable, for connecting to other devices.

The hardware configuration of the assessment evaluation device 1 is not limited to the configuration shown in FIG. 3A. For example, the assessment evaluation device 1 may include at least one of the input device 2 and/or the output device 3.

FIG. 3B shows the hardware configuration of the learning device 5. The learning device 5 includes a processor 51, a memory 52, and an interface 53 as hardware. The processor 51, memory 52 and interface 53 are connected to one another via a data bus 50.

The processor 51 functions as a controller (computing unit) for controlling the entire learning device 5 by executing a program stored in the memory 52. Examples of the processor 51 include a CPU, a GPU, a TPU and any other processor. The processor 51 may be configured by a plurality of processors.

The memory 52 is configured by various volatile memories and non-volatile memories such as a RAM, a ROM, a flash memory, and the like. Further, the memory 52 stores a program for executing a process executed by the learning device 5. A part of the information stored in the memory 52 may be stored in one or more external storage devices that can communicate with the learning device 5, or may be stored in a storage medium detachable from the learning device 5.

The interface 53 is one or more interfaces for electrically connecting the learning device 5 to other devices. Examples of these interfaces include a wireless interface, such as a network adapter, for transmitting and receiving data to and from other devices wirelessly, and a hardware interface, such as a cable, for connecting to other devices. Any input device and/or output device, or the like may be connected to the interface 53.

The hardware configuration of the learning device 5 is not limited to the configuration shown in FIG. 3B. For example, the learning device 5 may include at least one of an input device and/or an output device.

### (3) Functional Blocks

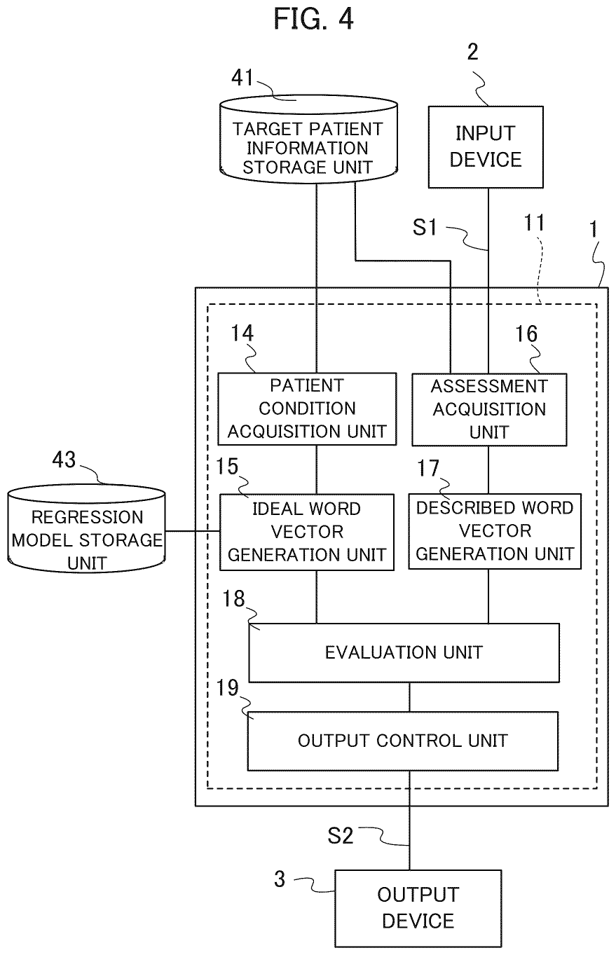

FIG. 4 is an example of functional blocks of the assessment evaluation device 1. The processor 11 of the assessment evaluation device 1 functionally includes a patient condition acquisition unit 14, an ideal word vector generation unit 15, an assessment acquisition unit 16, a described word vector generation unit 17, an evaluation unit 18, and an output control unit 19. In FIG. 4, blocks to exchange data with each other are connected by a solid line, but the combinations of blocks to exchange data with each other is not limited to FIG. 4. The same applies to the drawings of other functional blocks described below.

The patient condition acquisition unit 14 acquires information regarding the patient condition stored in the target patient information storage unit 41 associated with the patient ID of the target patient through the interface 13. In this case, the patient condition acquisition unit 14 converts the information regarding the patient condition acquired from the target patient information storage unit 41 into a vector format which matches the input format of the regression model, and supplies the patient condition represented as a vector format to the ideal word vector generation unit 15. The patient condition may be represented in a tensor format other than the vector format, instead of being represented by a vector format.

The ideal word vector generation unit 15 generates an ideal word vector based on the patient condition supplied from the patient condition acquisition unit 14. In this case, the ideal word vector generation unit 15 inputs the patient condition to one or more regression models built with reference to the regression model storage unit 43 and then acquires, from the regression models, the probabilities that N candidate words are ideal words, respectively. Then, for example, the ideal word vector generation unit 15 converts each of the probabilities for the N candidate words outputted by the regression models into 0 or 1 using a threshold value to thereby generate the ideal word vector in which the converted N values are used as elements of respective dimensions. In this case, “0” indicates that the target candidate word is not an ideal word, and “1” indicates that the target candidate word is an ideal word. In this way, for example, the ideal word vector is a vector whose elements are binary values indicative of the probabilities for respective candidate words outputted by the regression models. Details of the above-described binarization process will be described later.

In some embodiments, the ideal word vector generation unit 15 may use, as the elements of respective dimensions of the ideal word vector, the probabilities for respective candidate words outputted by the regression models as they are, instead of setting each element of the ideal word vector to 0 or 1 through binarization processing. In this case, the ideal word vector becomes a vector representing the probabilities that the N candidate vectors are ideal words, respectively. The ideal word vector generation unit 15 supplies the ideal word vector to the evaluation unit 18.

The assessment acquisition unit 16 acquires text information (i.e., assessment information) indicating the target assessment of evaluation. In this instance, for example, the assessment acquisition unit 16 acquires the input signal S1 indicating the target assessment of evaluation from the input device 2 via the interface 13, and generates the assessment information based on the input signal S1. In another example, the assessment acquisition unit 16 acquires assessment information included in the nursing records of the target patient from the target patient information storage unit 41. Then, the assessment acquisition unit 16 supplies the acquired assessment information to the described word vector generation unit 17.

The described word vector generation unit 17 generates the described word vector of the target assessment of evaluation. Here, the described word vector is represented by the same space as the coordinate system of the ideal word vector, and it is a word vector indicating the presence or absence of each of the candidate words in the target assessment of the evaluation by 0 or 1 for each dimension. In the present example embodiment, any element “0” of the described word vector indicates that the corresponding candidate word does not exist, and any element “1” of the described word vector indicates that the corresponding candidate word exists. The word vector generation unit 17 supplies the described word vector to the evaluation unit 18.

The evaluation unit 18 determines the evaluation of the target assessment based on the ideal word vector supplied from the ideal word vector generation unit 15 and the described word vector supplied from the described word vector generation unit 17. In this case, the evaluation unit 18 calculates the degree of similarity between the ideal word vector and the described word vector, and then determines the evaluation of the target assessment based on the calculated degree of similarity. Examples of the degree of similarity in this case include Jacket coefficient, Minkowski distance (including Manhattan distance, Euclidean distance, and Chebyshev distance), cosine similarity, and the like. The evaluation of the target assessment may be a binary value indicating the quality of the target assessment, a level indicating the quality of the target assessment on a scale of three or more, a score normalized to a predetermined value range (e.g., 0 to 100), or a combination thereof. The evaluation unit 18 supplies the determined evaluation result of the target assessment to the output control unit 19.

The output control unit 19 outputs information regarding the evaluation of the target assessment determined by the evaluation unit 18. In this instance, the output control unit 19 generates an output signal S2 for displaying a display screen image representing the evaluation result of the target assessment, and supplies the output signal S2 to the output device 3 through the interface 13, thereby causing the output device 3 to display the above-described display screen image. Specific display examples will be described later.

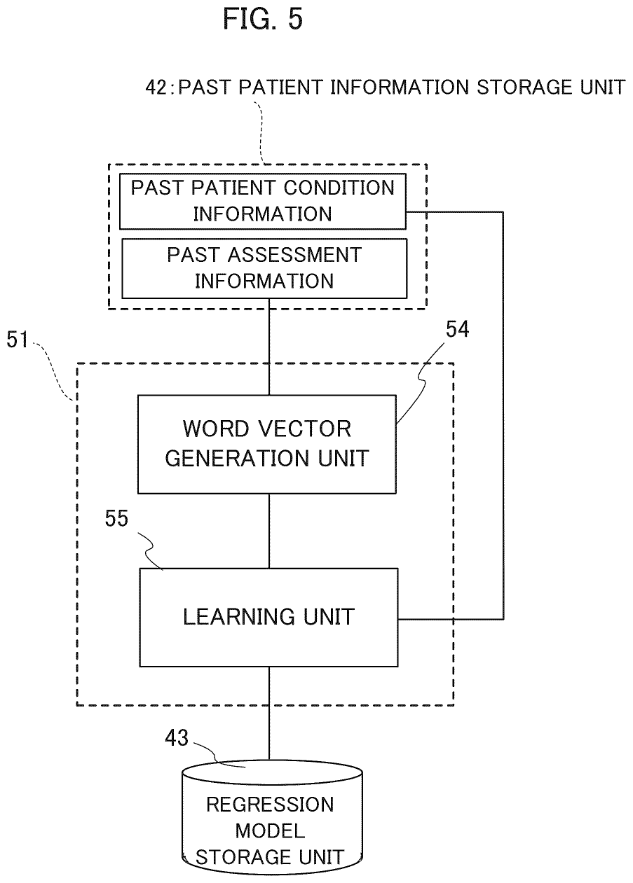

FIG. 5 is an example of functional blocks of the learning device 5. The processor 51 of the learning device 5 functionally includes a word vector generation unit 54 and a learning unit 55.

The word vector generation unit 54 acquires the past assessment information used for learning from the regression model storage unit 43 through the interface 53. Then, the word vector generation unit 54 generates a word vector based on the acquired past assessment information. In this case, for each assessment of the past patients, the word vector generation unit 54 generates a word vector indicating whether or not each of the N candidate words is included therein. The word vector in this case is represented by the same space (the same coordinate system) as the described word vector and the ideal word vector, and it is a word vector indicating the presence or absence of each candidate word in the assessment generated in the past by 0 or 1 for each dimension. Then, the word vector generation unit 54 supplies the word vector generated for each past assessment to the learning unit 55.

The learning unit 55 trains the regression model(s) based on the word vectors supplied from the word vector generation unit 54 and the past patient condition information obtained from the regression model storage unit 43 via the interface 53, and stores the parameters of the regression models obtained through the training (learning) in the regression model storage unit 43. In this case, the learning unit 55 converts the patient condition of each past patient into a vector format and uses the patient condition of each past patient represented in the vector format as an input to a regression model. The vector representing the patient condition of each past patient is a vector represented by the same coordinate system (the same feature space) as the vector representing the patient condition used by the ideal word vector generation unit 15. The learning unit 55 recognizes a correct answer (i.e., an ideal output) to be outputted by the regression model based on the word vector generated by the word vector generation unit 54. Then, the learning unit 55 determines the parameters of the regression model based on the set of the input to the regression model and the correct answer to be outputted. In this case, for example, the learning unit 55 determines the parameters of the regression model so that the error (loss) between the output from the regression model according to the input and the correct answer corresponding to the input to the regression model is minimized. The algorithm for determining parameters to minimize the loss may be any learning algorithm used in machine learning, such as gradient descent and error back propagation.

For example, each component of the patient condition acquisition unit 14, the ideal word vector generation unit 15, the assessment acquisition unit 16, the described word vector generation unit 17, the evaluation unit 18, and the output control unit 19 described in FIG. 4 can be realized by the processor 11 executing a program. Each component of the word vector generation unit 54 and the learning unit described in FIG. 5 can be realized by the processor 11 executing a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA (Field-Programmable Gate Array) and microcontrollers. In this case, the integrated circuit may be used to realize a program for configuring each of the above-described components. Further, at least a part of the components may be configured by a ASSP (Application Specific Standard Produce), ASIC (Application Specific Integrated Circuit) and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology. The above is the same in other example embodiments described later.

### (4) Examples of Internal Processing

Next, a description will be given of specific examples of the internal processing that is executed by the assessment evaluation device 1 and the learning device 5 in the first example embodiment.

First, when training a regression model, N candidate words are determined. For example, the candidate words are determined to be N (N is an integer of 2 or more) words having the highest frequency of appearance among the past patient assessments stored in the past patient information storage unit 42. In this case, the words of the past patient assessments are extracted by morphological analysis, and N candidate words are determined through frequency aggregation of the extracted words. The candidate words are not limited to being determined by this method, and may be determined to be important keywords in the assessment based on any technique (including any heuristic technique). In this specific example, it is assumed that three words (N=3) of “Breath”, “Sleep”, and “Fatigue” are determined to be candidate words.

Then, the features of the patient condition to be inputted to the regression models (in detail, the features corresponding to each axis of the feature space representing the patient condition) are determined. Here, as an example, the patient condition is assumed to be represented by a two-dimensional coordinate system with an axis corresponding to “Pulse” and an axis corresponding to “Disease name”. The “Pulse” and “Disease name” are quantified based on a predetermined rule. Hereafter, “X” is a variable representing “Pulse” and “Y” is a variable representing “Disease name”. Thus, the patient condition is represented as a vector (X, Y) in the two dimensional coordinate system described above.

Then, the learning device 5 trains regression models based on the past patient condition information and past assessment information stored in the regression model storage unit 43. In this case, for example, the learning device 5 trains a total of three regression models configured to output the probability of each candidate word as an ideal word. In this instance, the regression model for the candidate word “Breath” outputs a posterior probability “P (Breath|X, Y)” according to the patient condition (X, Y) with respect to the appearance of “Breath” in the target assessment. Similarly, the regression model for the candidate word “Sleep” outputs a posterior probability “P (Sleep|X, Y)” according to the patient condition (X, Y) regarding the appearance of “Sleep” in the target assessment, and the regression model for the candidate word “Fatigue” outputs a posterior probability “P (Fatigue|X, Y)” according to the patient condition (X, Y) regarding the appearance of “Fatigue” in the target assessment.

Then, in the evaluation stage, the assessment evaluation device 1 acquires the patient condition of the target patient and generates an ideal word vector by utilizing the learned regression models. For example, when “Pulse” of the target patient is set to “X1” and “Disease name” is set to “Y1”, the following probability vector is calculated.

P(P(Breath|X1,Y1),P(Sleep|X1,Y1),P(Fatigue|X1,Y1))

Hereafter, it is assumed that P (Breath|X1, Y1) is “0.8”, P (Sleep|X1, Y1) is “0.1”, and P (Fatigue|X1, Y1) is “0.6”, and therefore the probability vector P (0.8, 0.1, 0.6) is obtained.

Next, the assessment evaluation device 1 generates an ideal word vector based on the probability vector P. In this case, the assessment evaluation device 1 sets a threshold value to be compared with each element of the probability vector P, and sets elements equal to or larger than the threshold value to 1 while setting elements smaller than the threshold value to 0. For example, when the threshold value is set to “0.5”, an ideal word vector (1, 0, 1) is obtained from the probability vector P (0.8, 0.1, 0.6). This ideal word vector indicates that “Breath” and “Fatigue” fall under ideal words (i.e., the words that is necessary to be included in the assessment), and “Sleep” does not fall under the ideal word.

Here, the above-described threshold value may be set to a common predetermined value stored in the memory 12 or the storage device 4 in advance, or may be set to a value in accordance with the candidate word. In the latter case, for example, the assessment evaluation device 1 may determine a threshold value, to be compared with the vector element corresponding to each candidate word, in accordance with the frequency at which each candidate word appears in the assessments of the past patients stored in the past patient information storage unit 42. In this case, for example, the assessment evaluation device 1 decreases the threshold value corresponding to a candidate word with an increase in the above-mentioned frequency of the candidate word, based on a predetermined equation or table information prepared in advance. Thus, the more frequently used words are, the easier they are adopted as ideal words.

In some embodiments, the assessment evaluation device 1 regards the probability vector P as a vector obtained by generalizing the elements of the ideal word vector to continuous quantities, and uses the probability vector P as an ideal word vector. In this case, the assessment evaluation device 1 does not perform the binarization process using the above-described threshold value.

Next, the assessment evaluation device 1 generates the described word vector based on the target assessment of the evaluation. As an example, it is herein assumed that the target assessment of evaluation indicates the following sentence.

“The cause of fatigue is considered to be insufficient sleep.”

In this case, the assessment evaluation device 1 extracts the words (“sleep” and “fatigue”) described in the target assessment of the evaluation by morphological analysis and generates the described word vector by determining whether or not the extracted words fall under each candidate word (“Breath”, “Sleep” and “Fatigue”). Here, since the words corresponding to the candidate words “Sleep” and “Fatigue” are extracted and the word corresponding to the candidate word “Breath” are not extracted, the described word vector becomes (0, 1, 1).

Then, the assessment evaluation device 1 calculates the degree of similarity between the ideal word vector (1, 0, 1) and the described word vector (0, 1, 1), and then determines the evaluation of the target assessment of the evaluation, based on the calculated degree of similarity.

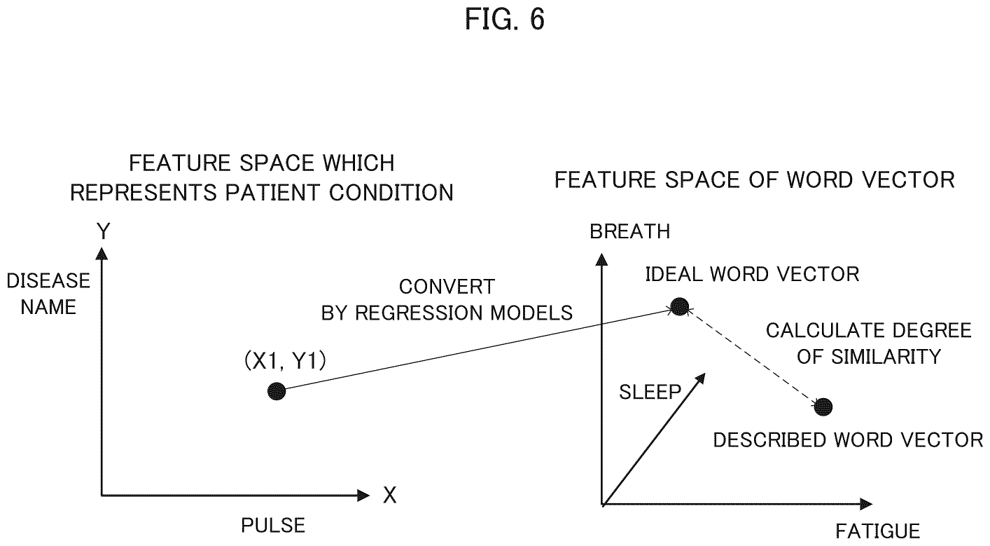

FIG. 6 illustrates the processing outline of the assessment evaluation device 1 in a specific example. Here, the left figure is a coordinate system which represents the feature space of the patient condition, and the right figure is a coordinate system which represents the feature space of the ideal word vector and the described word vector. The assessment evaluation device 1 uses regression models to convert (X1, Y1), which corresponds to a combination of the “Pulse” and “Disease name” indicative of the patient condition of the target patient, into an ideal word vector. In addition, the assessment evaluation device 1 generates the described word vector by morphological analysis on the target assessment of the evaluation. Then, the assessment evaluation device 1 calculates the degree of similarity between the ideal word vector and the described word vector and determines the evaluation based on the calculated degree of similarity.

### (5) Display Example

Next, a description will be given of display examples (first display example to third display example) which the output control unit 19 of the assessment evaluation device 1 displays on the output device 3.

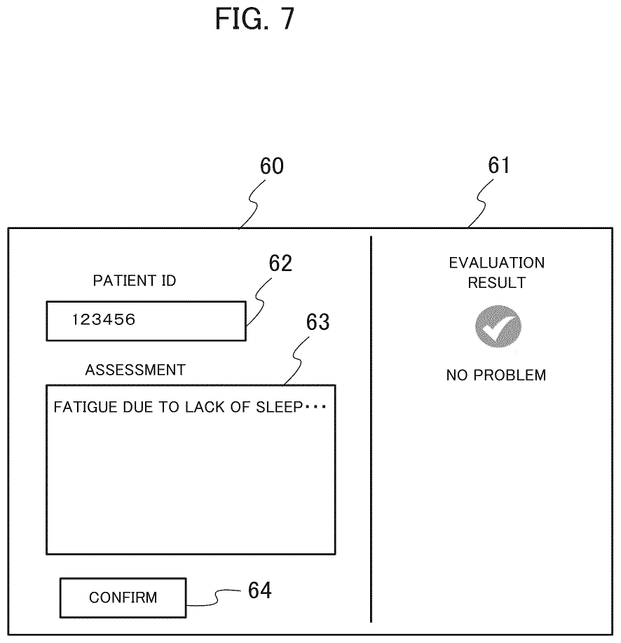

FIG. 7 is a first display example relating to the evaluation of the assessment. In the first display example, the output control unit 19 presents the evaluation result of the assessment specified by the input of the patient ID to the user. The output control unit 19 generates an output signal S2 based on the input signal S1 or the like generated by the input device 2, and then transmits the output signal S2 to the output device 3 to cause the output device 3 to display the display screen image shown in FIG. 7.

On the display screen image according to the first display example, there are provided an input-related display area 60, and an evaluation result display area 61. Then, on the input-related display area 60, the output control unit 19 provides a patient ID input field 62 for receiving input of the patient ID from a user, an assessment input field 63 for receiving input of the assessment for the patient ID specified in the patient ID input field 62 from a user, and a confirmation button 64 for instructing to evaluate the assessment inputted in the assessment input field 63. Upon detecting that the confirmation button 64 is selected, the output control unit 19 displays information indicating the evaluation result of the assessment inputted in the assessment input field 63. In the first display example, the output control unit 19 determines the quality (there is a problem or no problem) of the target assessment of the evaluation, and displays the evaluation result to the effect that the assessment is no problem on the evaluation result display area 61.

The calculation of the evaluation result in the first display example will be supplementally described. If the output control unit 19 detects that the confirmation button 64 has been selected, the patient condition acquisition unit 14 acquires the patient condition of the target patient from the target patient information storage unit 41 associated with the patient ID “123456” specified in the patient ID input field 62, and the ideal word vector generation unit 15 generates the ideal word vector based on the patient condition acquired by the patient condition acquisition unit 14. The assessment acquisition unit 16 acquires the text information inputted in the assessment input field 63 as assessment information regarding the target assessment of evaluation, and the described word vector generation unit 17 generates the described word vector by morphological analysis on the assessment information. Then, the evaluation unit 18 calculates the degree of similarity between the ideal word vector and the described word vector. Since the degree of similarity is equal to or greater than a predetermined threshold value, the evaluation unit 18 generates an evaluation result representing that there is no problem in the assessment, and supplies the evaluation result to the output control unit 19.

Thus, according to the first display example, the assessment evaluation equipment 1 can present to a user suitably the evaluation result of the assessment specified by input of the patient ID on a display screen image. In addition, for example, when the user is in a nurturing stage such as a training period, the assessment evaluation device 1 can present the evaluation result of the assessment entered by the user as feedback. Thus, according to the first display example, it is possible to support the education of the user. As a modification of the first display example, the assessment evaluation device 1 may provide, in the input-related display area 60, an input field for inputting the patient condition, and recognize the patient condition based on the input to the input field.

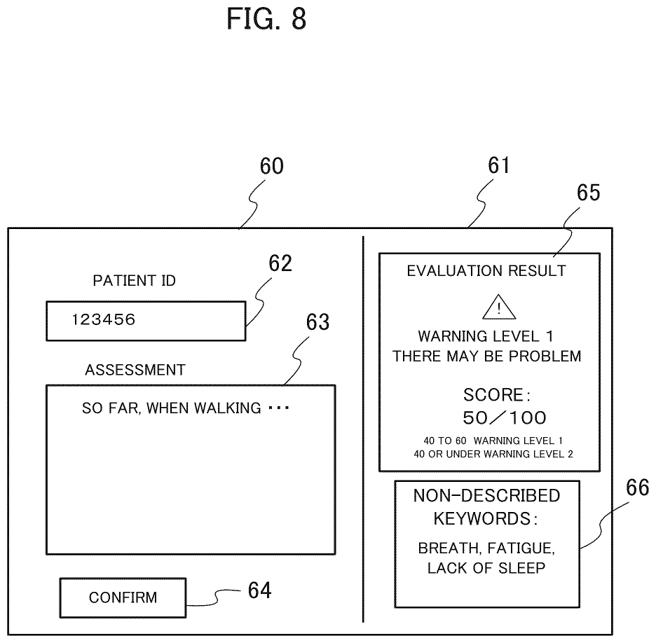

FIG. 8 is a second display example relating to the evaluation of the assessment. In the second display example, the output control unit 19 displays, as an evaluation of the assessment, a score and the level which indicate the quality of the assessment, and presents ideal words that are missing in the target assessment of the evaluation.

On the display screen image according to the second display example, there are provided an input-related display area 60, and an evaluation result display area 61. Then, the output control unit 19 displays the patient ID input field 62 described above, the assessment input field 63, and the confirmation button 64 on the input related display area 60. Further, upon detecting that the confirmation button 64 is selected, the output control unit 19 displays the evaluation result display field 65 and the non-described word display field 66 on the evaluation result display area 61.

On the evaluation result display field 65, the output control unit 19 displays, as the evaluation result, a score (50 in this case) and the level (warning level 1 in this case) of the score. Supplementary description will be given of the process executed by the evaluation unit 18 in this case. In the second display example, the evaluation unit 18 calculates, as the evaluation result, a score obtained by normalizing the degree of similarity between the ideal word vector and the described word vector to range from 0 to 100. Then, by providing a plurality of threshold values for the calculated score, the evaluation unit 18 determines the level of the score. The highest value of the score is 100 and the lowest value thereof is 0. Further, in the level determination of the score, the evaluation unit 18 provides a first threshold value (60 in this case) for determining whether or not a warning is a necessary, a second threshold (40 in this case) for subdividing the warning level into two. Then, the evaluation unit 18 determines the calculated score falls under the warning level 1 since the calculated score is higher than the second threshold value while not exceeding the first threshold value. Then, the output control unit 19 displays the determination result of the score and the level on the evaluation result display field 65.

Further, in the non-described word display field 66, the output control unit 19 displays ideal words (keywords in the figure) that are not included in the target assessment of the evaluation. Specifically, among the ideal words (i.e., the candidate words corresponding to the elements “1” of the ideal word vector), the output control unit 19 displays words which are not described in the target assessment of the evaluation in the non-described word display field 66. Thus, the output control unit 19 can assist the user to grasp the keywords that are estimated to be missing in the assessment.

In some embodiments, instead of displaying the ideal words which are not described, or in addition to the process, the output control unit 19 may display words which do not fall under ideal words among the words (which may be limited to candidate words) included in the target assessment of the evaluation, as unnecessarily-described words. Thus, the output control unit 19 can notify the user that there is an extra description in the target assessment of the evaluation.

As described above, even in the second display example, the assessment evaluation device 1 can present the evaluation result of the assessment entered with the patient ID on the display screen image to the user. In addition, when the user is in the nurturing stage such as the training period, the assessment evaluation device 1 presents the detailed evaluation result of the assessment entered by the user and the words which are missing in the assessment as feedback, and can support the education of the user.

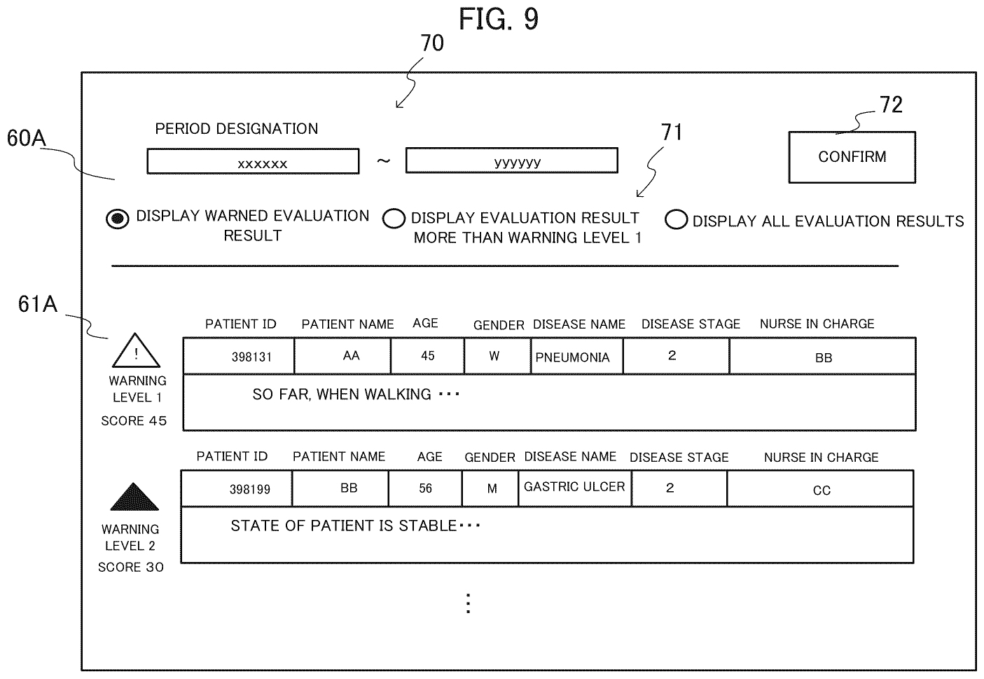

FIG. 9 is a third display example relating to the assessment evaluation. In the third display example, the output control unit 19 collectively performs evaluation for a plurality of target patient assessment stored in the target patient information storage unit 41, and displays the evaluation result.

On the display screen image according to the third display example, there are provided an input-related display area 60A and an evaluation-result display area 61A. Then, the output control unit 19 displays, on the input-related display area 60A, a period designation input field 70, a display selection field 71, and a confirmation button 72. Here, the period designation input field 70 is an input field for the user to specify the period in which the target assessment of evaluation is generated. Further, the display selection field 71 is a field for receiving the level of the score to specify the assessments whose evaluation results are displayed on the evaluation result display area 61A and as an example it is herein an input field according to a radio-button format. It is herein assumed that the score and the level of the score are calculated by the same calculation method as the second display example. The confirmation button 72 is a button for receiving instructions to evaluate the assessment from the user.

Upon detecting that the confirmation button 72 is selected, the assessment evaluation device 1 evaluates the assessments generated in the period specified by the period designation input field 70. Then, among the generated evaluation results, the output control unit 19 displays the list of the target patients whose evaluation results are specified in the display selection field 71, on the evaluation result display area 61A. In the third display example, the output control unit 19 displays sets of nursing records and evaluation results regarding the target patients corresponding to warned evaluation results (that is, the evaluation results with the warning level 1 or the warning level 2) on the evaluation result display area 61A. The above nursing records include information regarding the patient ID, the patient name, the age, the gender, the patient name, the disease stage, the nurse in charge, and the assessment subject to assessment, and the above evaluation results include information regarding the sore and the level of the score.

Thus, according to the third display example, the assessment evaluation device 1 conducts collective evaluations of the assessments stored in the database or the like to thereby present the presence of the warned assessments to the user. As described above, according to each display example in the first example embodiment, it is possible to display the evaluation result regarding whether or not the assessment is correctly described, to thereby support the generation of the nursing records. Here, the support of the preparation of the nursing records includes the support of nurturing the user who makes the nursing records.

### (6) Processing Flow

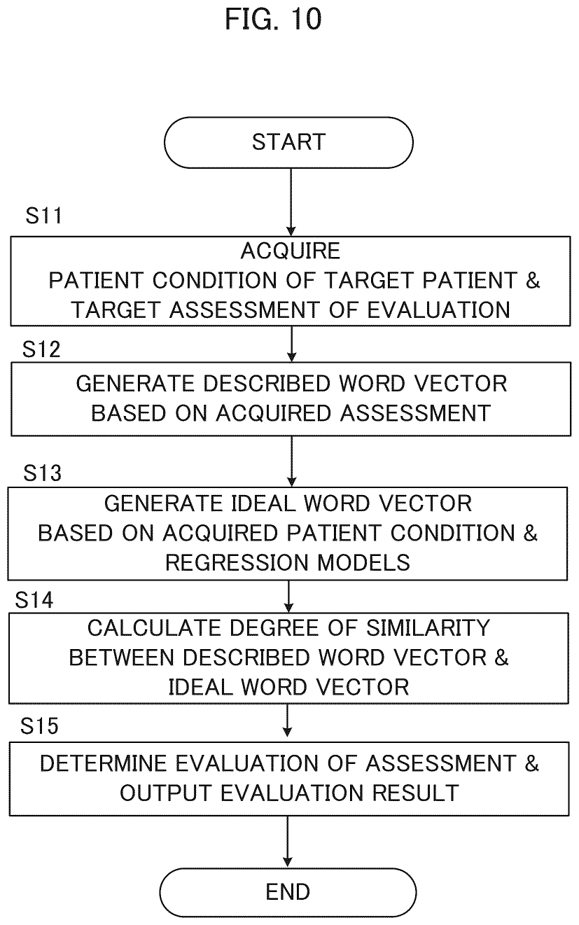

FIG. 10 is an example of a flowchart that is executed by the assessment evaluation device 1 in the first example embodiment.

First, the assessment evaluation device 1 acquires the patient condition of the target patient and target assessment of evaluation (step S11). For example, the assessment evaluation device 1 acquires the information from the target patient information storage unit 41 of the storage device 4 or the input device 2. Then, based on the assessment acquired at step S11, the assessment evaluation device 1 generates a described word vector (step S12). In addition, the assessment evaluation device 1 generates an ideal word vector based on the patient condition represented in a vector format or the like and learned regression models (step S13). The order of the processes at step S12 and the step S13 may be reversed.

Next, the assessment evaluation device 1 calculates the degree of similarity between the described word vector calculated at step S12 and the ideal word vector calculated at step S13 (step S14). Then, based on the degree of similarity calculated at step S14, the assessment evaluation device 1 determines the evaluation of the target assessment, and outputs the determined evaluation result by the output device 3 (step S15).

### (7) Modifications

The evaluation unit 18 may determine the evaluation of the assessment based on the ideal word vector and described word vector, without performing similarity calculation.

For example, the evaluation unit 18 may determine the above-mentioned evaluation of the assessment using an evaluation model, which is configured to output an evaluation result (classification result regarding the quality or the score) of a target assessment when an ideal word vector and a described word vector regarding the target assessment is inputted thereto. In this case, the evaluation model is a machine learning model, such as a neural network, and is previously trained to output the evaluation result of a target assessment when the ideal word vector and the described word vector regarding the target assessment are inputted thereto, and the learned parameters are stored in the storage device 4 or the memory 12 or the like. With such an evaluation model, the evaluation unit 18 can also determine the evaluation of the target assessment.

### Second Example Embodiment

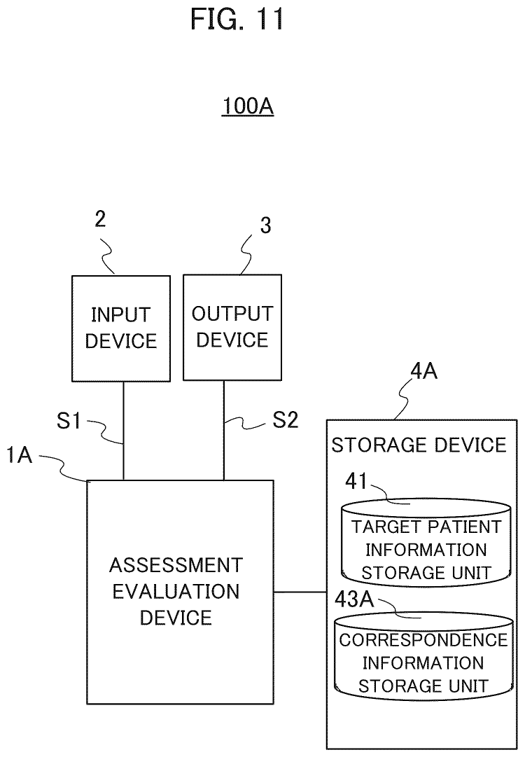

FIG. 11 shows a schematic configuration of an assessment support system 100A according to a second example embodiment. The assessment evaluation device 1A of the assessment support system 100A differs from the assessment evaluation device 1 according to the first example embodiment in that it generates the ideal word vector by using information indicating a correspondence (i.e., a rule of correspondence) between a patient condition and ideal words that is necessary to be included in the assessment of the patient condition, instead of generating the ideal word vector based on regression models. Hereinafter, the same components as in the first example embodiment are appropriately denoted by the same reference numerals, and a description thereof will be omitted.

The storage device 4A functionally includes a target patient information storage unit 41 and a correspondence information storage unit 43A. The correspondence information storage unit 43A stores the correspondence information indicating the correspondence between the patient condition and the ideal words. For example, the correspondence information is table information (look-up table) indicating ideal words to be set for each possible patient condition. The assessment evaluation device 1A recognizes ideal words based on the patient condition and the correspondence information, and converts the recognized ideal words into an ideal word vector. The correspondence information may be information in which an ideal word vector is directly associated with each possible patient condition. The correspondence information is generated in advance, for example, based on medical and nursing knowledge.

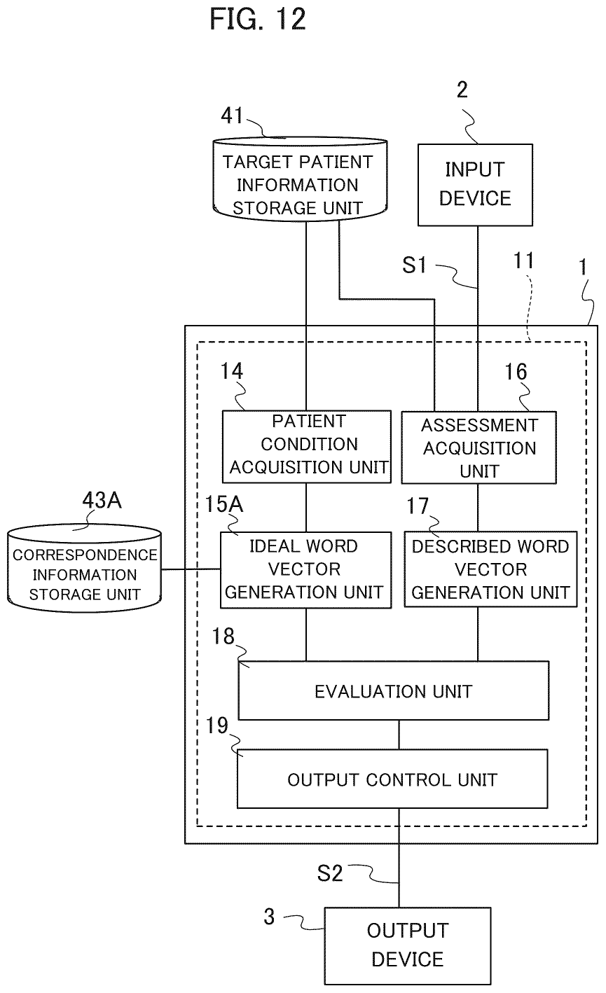

FIG. 12 is an example of functional blocks of the assessment evaluation device 1A. The assessment evaluation device 1A is equipped with the same hardware configuration as the hardware configuration of the assessment evaluation device 1 shown in FIG. 3A. The processor 11 of the assessment evaluation device 1A functionally includes a patient condition acquisition unit 14, an ideal word vector generation unit 15A, an assessment acquisition unit 16, a described word vector generation unit 17, an evaluation unit 18, and an output control unit 19. The patient condition acquisition unit 14, the assessment acquisition unit 16, described word vector generation unit 17, the evaluation unit 18, and the output control unit 19 executes the same processing as in the first example embodiment.

The ideal word vector generation unit 15A generates an ideal word vector based on the patient condition information supplied from the patient condition acquisition unit 14. In this instance, based on the correspondence information stored in the correspondence information storage unit 43A, the ideal word vector generation unit 15A generates an ideal word vector corresponding to the patient condition information represented in the vector format. Then, the ideal word vector generation unit 15A supplies the ideal word vector to the evaluation unit 18. Thereafter, the evaluation unit 18 generates an evaluation result based on the degree of similarity between the ideal word vector and the described word vector generated by the described word vector generation unit 17, and the output control unit 19 outputs the evaluation result by the output device 3.

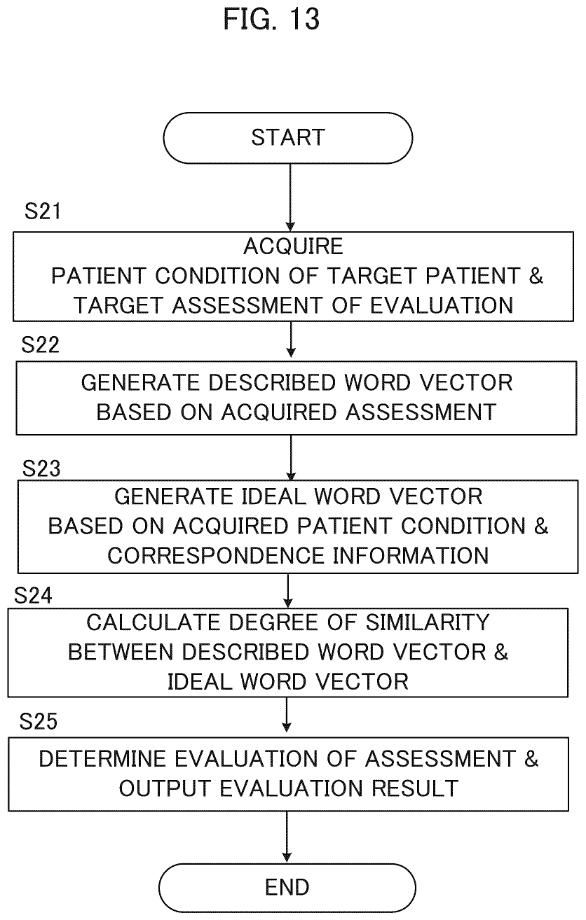

FIG. 13 is an example of the flowchart that is executed by the assessment evaluation device 1A in the second example embodiment.

First, the assessment evaluation device 1A acquires the patient condition of the target patient and the target assessment of evaluation (step S21). Then, the assessment evaluation device 1 generates a described word vector based on the target assessment acquired at step S21 (step S22). Further, the assessment evaluation device TA generates the ideal word vector based on the patient condition and the correspondence information stored in the correspondence information storage unit 43A (step S23). The order of the processes at step S22 and step S23 may be reversed. Next, the assessment evaluation device TA calculates the degree of similarity between the described word vector calculated at step S22 and the ideal word vector calculated at step S23 (step S24). Then, based on the degree of similarity calculated at step S24, the assessment evaluation device TA determines the evaluation of the target assessment of the evaluation, and outputs the determined evaluation result by the output device 3 (step S25).

Thus, the assessment evaluation device TA according to the second example embodiment, like the assessment evaluation device 1 according to the first example embodiment, can determine the evaluation of the target assessment.

### Third Example Embodiment

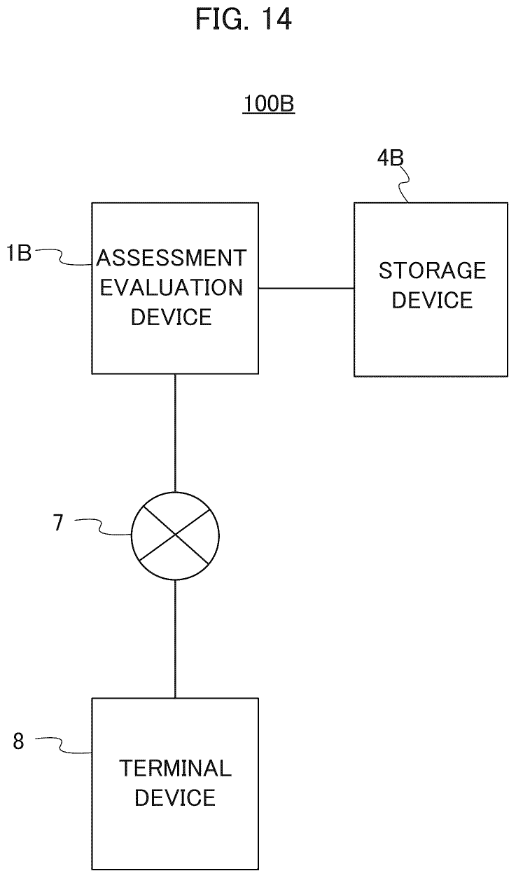

FIG. 14 shows a schematic configuration of an assessment support system 100B according to a third example embodiment. The assessment support system 100B according to the third example embodiment is a system of a server client model, and an assessment evaluation device 1B functioning as a server device performs the same process as the assessment evaluation device 1 according to the first example embodiment or the assessment evaluation device TA according to the second example embodiment does. Hereinafter, the same components as in the first example embodiment or the second example embodiment are appropriately denoted by the same reference numerals, and a description thereof will be omitted.

The assessment support system 100B mainly includes an assessment evaluation device 1B that functions as a server, a storage device 4B that stores data stored in the storage device 4 according to the first example embodiment or the storage device 4A according to the second example embodiment, and a terminal device 8 that functions as a client. The assessment evaluation device 1B and the terminal device 8 performs data communication with each other via the network 7.

The terminal device 8 is a terminal equipped with an input function, a display function, and a communication function, and functions as the input device 2 and the output device 3 shown in FIG. 1. Examples of the terminal device 8 include a personal computer, a tablet-type terminal, and a PDA (Personal Digital Assistant). The terminal device 8 transmits an input signal or the like based on the user input to the assessment evaluation device 1B.

The assessment evaluation device 1B is equipped with the same hardware configuration and functional block configuration as the assessment evaluation device 1 according to the first example embodiment or the assessment evaluation device TA according to the second example embodiment. Then, the assessment evaluation device TA receives an input signal or the like from the terminal device 8 via the network 7, and evaluates the assessment of the target patient based on the received information. Based on a request from the terminal device 8, the assessment evaluation device 1B transmits an output signal indicating the information regarding the evaluation result described above to the terminal device 8 via the network 7. That is, in this case, the terminal device 8 functions as the output device 3 in the first example embodiment or the second example embodiment. Thus, the assessment evaluation device 1B suitably provides the user of the terminal device 8 with information regarding the assessment results.

### Fourth Example Embodiment

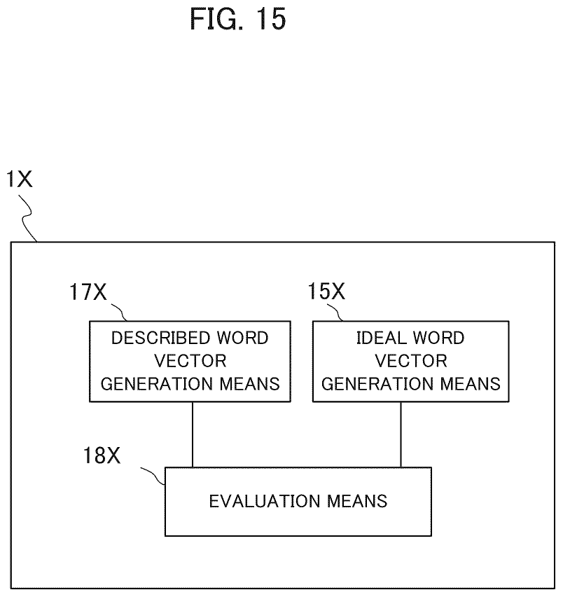

FIG. 15 is a block diagram of an assessment evaluation device 1X according to a fourth example embodiment. The assessment evaluation device 1X mainly includes a described word vector generation means 17X, an ideal word vector generation means 15X, and an evaluation means 18X. The assessment evaluation device 1X may be configured by a plurality of devices.

The described word vector generation means 17X is configured to generate, based on a nursing assessment of a target patient whose nursing records are to be prepared, a described word vector which is a word vector obtained by vectorizing whether or not each of predetermined candidate words is described in the nursing assessment. Examples of the described word vector generation means 17X include the word vector generation unit 17 described in the first example embodiment to the third example embodiment.

The ideal word vector generation means 15X is configured to generate an ideal word vector that is an ideal of the word vector based on a patient condition of the target patient. Examples of the ideal word vector generation means 15X include the ideal word vector generation unit 15 in the first example embodiment or the third example embodiment, and the ideal word vector generation unit 15A in the second example embodiment or the third example embodiment.

The evaluation means 18X is configured to determine an evaluation of the nursing assessment based on the described word vector and the ideal word vector. Examples of the evaluation means 18X include the evaluation unit 18 in the first example embodiment to the third example embodiment.

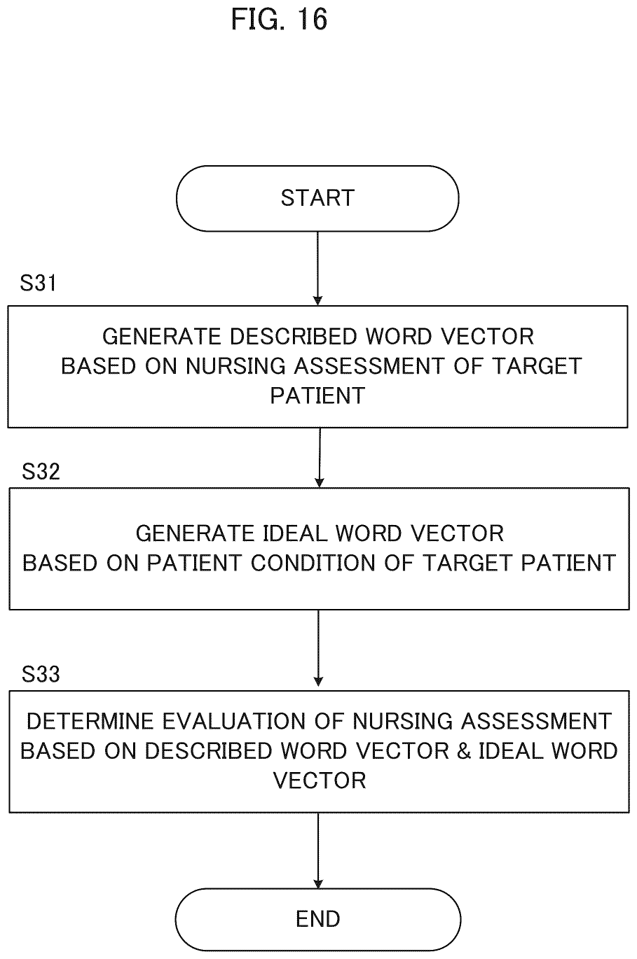

FIG. 16 is an example of the flowchart that is executed by the assessment evaluation device 1X in the fourth example embodiment. The described word vector generation means 17X generates, based on a nursing assessment of a target patient whose nursing records are to be prepared, a described word vector which is a word vector obtained by vectorizing whether or not each of predetermined candidate words is described in the nursing assessment (step S31). The ideal word vector generation means 15X generates an ideal word vector that is an ideal of the word vector based on a patient condition of the target patient (step S32). The evaluation means 18X determines an evaluation of the nursing assessment based on the described word vector and the ideal word vector (step S33).

According to the fourth example embodiment, the assessment evaluation device 1X is capable of determining an evaluation of a nursing assessment of a target patient.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments described above (including modifications, the same applies hereinafter) can be described as, but not limited to, the following Supplementary Notes.

### [Supplementary Note 1]

An assessment evaluation device comprising:
a described word vector generation means configured to generate, based on a nursing assessment of a target patient whose nursing records are to be prepared, a described word vector which is a word vector obtained by vectorizing whether or not each of predetermined candidate words is described in the nursing assessment;
an ideal word vector generation means configured to generate an ideal word vector that is an ideal of the word vector based on a patient condition of the target patient; and
an evaluation means configured to determine an evaluation of the nursing assessment based on the described word vector and the ideal word vector.

### [Supplementary Note 2]

The assessment evaluation device according to Supplementary Note 1,
wherein the evaluation means is configured to
   calculate a degree of similarity between the described word vector and the ideal word vector and
   determine the evaluation based on the degree of similarity.

### [Supplementary Note 3]

The assessment evaluation device according to Supplementary Note 1 or 2,
wherein the ideal word vector generation means is configured to generate the ideal word vector based on a regression model and the patient condition, and
wherein the regression model is a model which learned a relation between
   the patient condition and
   whether or not each of the candidate words is necessary to be described in the nursing assessment.

### [Supplementary Note 4]

The assessment evaluation device according to Supplementary Note 3,
wherein the regression model is configured to output a probability as to whether or not each of the candidate words is necessary to be described in the nursing assessment, and
wherein the ideal word vector generation means is configured to generate the ideal word vector which is a probability vector of the probability.

### [Supplementary Note 5]

The assessment evaluation device according to Supplementary Note 3,
wherein the regression model is configured to output a probability as to whether or not each of the candidate words is necessary to be described in the nursing assessment, and
wherein the ideal word vector generation means is configured to generate the ideal word vector which is a vector of a value obtained by binarizing the probability.

### [Supplementary Note 6]

The assessment evaluation device according to Supplementary Note 1 or 2,
wherein the ideal word vector generation means is configured to generate the ideal word vector corresponding to the patient condition based on correspondence information indicating correspondence between the patient condition and ideal words that are ideal to be described in the nursing assessment.

### [Supplementary Note 7]

The assessment evaluation device according to any one of Supplementary Notes 1 to 6,
wherein the ideal word vector generation means is configured to acquire, as the patient condition, at least one of
   attribute information of the target patient,
   information regarding an examination result of the target patient, and/or
   the information regarding past nursing records of the target patient.

### [Supplementary Note 8]

The assessment evaluation device according to any one of Supplementary Notes 1 to 7,
further comprising an output control means configured to cause an output device to output a result of the evaluation.

### [Supplementary Note 9]

The assessment evaluation device according to Supplementary Note 8,
wherein the result of the evaluation includes at least one of
   a determination result of a quality of the nursing assessment,
   a score which represents the quality of the nursing assessment using a predetermined value range, and/or
   a level of the score.

### [Supplementary Note 10]

The assessment evaluation device according to Supplementary Note 8 or 9,
wherein the output control means is configured to cause the output device to output at least one of
   a word which is not described in the nursing assessment among ideal words which are ideal to be described in the nursing assessment, and/or
   a word which is not included in the ideal words among words described in the nursing assessment.

### [Supplementary Note 11]

An assessment evaluation method executed by including:
generating, based on a nursing assessment of a target patient whose nursing records are to be prepared, a described word vector which is a word vector obtained by vectorizing whether or not each of predetermined candidate words is described in the nursing assessment;
generating an ideal word vector that is an ideal of the word vector based on a patient condition of the target patient; and
determining an evaluation of the nursing assessment based on the described word vector and the ideal word vector.

### [Supplementary Note 12]

A storage medium storing a program executed by a computer, the program causing the computer to:
generate, based on a nursing assessment of a target patient whose nursing records are to be prepared, a described word vector which is a word vector obtained by vectorizing whether or not each of predetermined candidate words is described in the nursing assessment;
generate an ideal word vector that is an ideal of the word vector based on a patient condition of the target patient; and
determine an evaluation of the nursing assessment based on the described word vector and the ideal word vector.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### INDUSTRIAL APPLICABILITY

For example, it is used for the support of nursing records and the service on the education of nursing record preparation.

### DESCRIPTION OF REFERENCE NUMERALS

1. TA, 1B, 1X Assessment evaluation device
2 Input device
3 Output device
4 Storage device
5 Learning device
8 Terminal device
100, 100A, 100B Assessment support system

What is claimed is:

1. An assessment evaluation device comprising:
at least one memory configured to store instructions; and
at least one processor configured to execute the instructions to:
generate, based on a nursing assessment of a target patient whose nursing records are to be prepared, a described word vector which is a word vector obtained by vectorizing whether or not each of predetermined candidate words is described in the nursing assessment;
generate an ideal word vector that is an ideal of the word vector based on a patient condition of the target patient; and
determine an evaluation of the nursing assessment based on the described word vector and the ideal word vector.

2. The assessment evaluation device according to claim 1,
wherein the at least one processor is configured to execute the instructions to
calculate a degree of similarity between the described word vector and the ideal word vector and
determine the evaluation based on the degree of similarity.

3. The assessment evaluation device according to claim 1,
wherein the at least one processor is configured to execute the instructions to generate the ideal word vector based on a regression model and the patient condition, and
wherein the regression model is a model which learned, by machine learning, a relation between
the patient condition and
whether or not each of the candidate words is necessary to be described in the nursing assessment.

4. The assessment evaluation device according to claim 3,
wherein the regression model is configured to output a probability as to whether or not each of the candidate words is necessary to be described in the nursing assessment, and
wherein the at least one processor is configured to execute the instructions to generate the ideal word vector which is a probability vector of the probability.

5. The assessment evaluation device according to claim 3,
wherein the regression model is configured to output a probability as to whether or not each of the candidate words is necessary to be described in the nursing assessment, and
wherein the at least one processor is configured to execute the instructions to generate the ideal word vector which is a vector of a value obtained by binarizing the probability.

6. The assessment evaluation device according to claim 1,
wherein the at least one processor is configured to execute the instructions to generate the ideal word vector corresponding to the patient condition based on correspondence information indicating correspondence between the patient condition and ideal words that are ideal to be described in the nursing assessment.

7. The assessment evaluation device according to claim 1,
wherein the at least one processor is configured to execute the instructions to acquire, as the patient condition, at least one of
attribute information of the target patient,
information regarding an examination result of the target patient, and/or
the information regarding past nursing records of the target patient.

8. The assessment evaluation device according to claim 1,
wherein the at least one processor is configured to further execute the instructions to cause an output device to output a result of the evaluation.

9. The assessment evaluation device according to claim 8,
wherein the result of the evaluation includes at least one of
a determination result of a quality of the nursing assessment,
a score which represents the quality of the nursing assessment using a predetermined value range, and/or
a level of the score.

10. The assessment evaluation device according to claim 8,
wherein the at least one processor is configured to execute the instructions to cause the output device to output at least one of
a word which is not described in the nursing assessment among ideal words which are ideal to be described in the nursing assessment, and/or
a word which is not included in the ideal words among words described in the nursing assessment.

11. An assessment evaluation method executed by including:
generating, based on a nursing assessment of a target patient whose nursing records are to be prepared, a described word vector which is a word vector obtained by vectorizing whether or not each of predetermined candidate words is described in the nursing assessment;
generating an ideal word vector that is an ideal of the word vector based on a patient condition of the target patient; and
determining an evaluation of the nursing assessment based on the described word vector and the ideal word vector.

12. A non-transitory computer readable storage medium storing a program executed by a computer, the program causing the computer to:
generate, based on a nursing assessment of a target patient whose nursing records are to be prepared, a described word vector which is a word vector obtained by vectorizing whether or not each of predetermined candidate words is described in the nursing assessment;
generate an ideal word vector that is an ideal of the word vector based on a patient condition of the target patient; and
determine an evaluation of the nursing assessment based on the described word vector and the ideal word vector.
